(19) United States
(12) Patent Application Publication
(10) Pub. No.: US 2025/0195253 A1
(43) Pub. Date: Jun. 19, 2025

(54) Non-Invasive Fracture Stabilizing Orthosis

(71) Applicant: The Government of the United States, as Represented by the Secretary of the Army, Fort Detrick, MD (US)

(72) Inventors: William Brett Johnson, San Antonio, TX (US); Walter Lee Childers, San Antonio, TX (US)

(21) Appl. No.: 19/066,693

(22) Filed: Feb. 28, 2025

Related U.S. Application Data
(63) Continuation-in-part of application No. PCT/US2023/032452, filed on Sep. 12, 2023.
(60) Provisional application No. 63/405,537, filed on Sep. 12, 2022.

(51) Int. Cl. A61F 5/058; A61F 5/01

(52) U.S. Cl. CPC A61F 5/05825; A61F 5/0111; A61F 5/0585

(57) ABSTRACT

A fracture stabilization orthosis includes a fabric or fabric wrap and a plurality of struts, each strut comprising two or more sheets of a material conformable to contours of a leg and insertable into a sleeve or pocket on the fabric or fabric wrap. The fabric or fabric wrap is shaped so that when it is wrapped around a limb it forms a frustum.

This is a Continuation-in-Part of PCT international application PCT/US2023/032452 filed on 12 Sep. 2023 and claims priority to U.S. Ser. No. 63/405,527 filed in the U.S. Patent and Trademark Office on 12 Sep. 2022, the entirety of which is incorporated herein by reference.

### FIELD OF INVENTION

The present invention is directed to a non-invasive fracture stabilization orthosis. The stabilization orthosis may help stabilize a limb with a bone fracture, for example, tibia and fibula bone fractures.

### BACKGROUND OF INVENTION

There is a need for a non-invasive fracture stabilization orthosis that can be used for prolonged care, for example, in an austere environment or an environment in which medical help is not immediately available.

Prolonged care may have several deleterious impacts on a medical or military unit. For example, leg bone fractures require vehicle transport, and evacuation may take days during which teams must remain mobile. The use of a litter for an injured person may take four people to carry, thereby removing five people from essential tasks. Furthermore, litter carry teams result in concentrating personnel in one area and are more exposed to adversarial threats. Thus, there remains a need for a fracture stabilization orthosis that can enable increased independent mobility for injured people having bone fractures.

### SUMMARY OF INVENTION

The invention provides in a first embodiment a fracture stabilization orthosis comprising a fabric or fabric wrap and a plurality of struts, each strut insertable into a sleeve or pocket on the fabric or fabric wrap. The fabric or fabric wrap is shaped so that when it is wrapped around a limb it forms a frustum.

The invention provides in a second embodiment further to any of the previous embodiments an orthosis in which the fabric or fabric wrap comprises an elastic material.

The invention provides in a third embodiment further to any of the previous embodiments an orthosis in which each strut comprises two or more layered sheets of material.

The invention provides in a fourth embodiment further to any of the previous embodiments an orthosis in which each strut comprises aluminum, titanium, carbon fiber laminate, fiberglass laminate, or KEVLAR® laminate.

The invention provides in a fifth embodiment further to any of the previous embodiments an orthosis further comprising two pieces of foam on an inner surface of the fabric or fabric wrap, so that when the orthosis is wrapped around a lower leg, each foam piece rests along an opposing side of the tibia crest.

The invention provides in a sixth embodiment further to any of the previous embodiments an orthosis in which the frustum has a pitch of about 0.6 to about 0.7, for example, about 0.66.

The invention provides in a seventh embodiment further to any of the previous embodiments an orthosis further comprising a footplate connected to a bottom of the fabric or fabric wrap. The footplate may be foldable to substantially cover a top surface of a foot.

The invention provides in a first method embodiment a method of stabilizing a fracture comprising providing a fracture stabilization orthosis according to any of the previous embodiments and wrapping the fracture stabilization orthosis around a limb of a person having a bone fracture. The bone fracture may be a tibia or fibula bone fracture.

### BRIEF DESCRIPTION OF THE DRAWINGS

FIG. 1 is a perspective view of a fracture stabilization orthosis according to an embodiment of the present invention.

FIG. 2 is a flat front perspective view of a fracture stabilization orthosis according to an embodiment of the present invention.

FIG. 3 is an isometric view of a fracture stabilization orthosis according to an embodiment of the present invention.

FIG. 4 is a view of a fracture stabilization orthosis showing a strut comprising layered sheets or strips being insertable into a pocket or sleeve according to an embodiment of the present invention.

FIG. 5 is a perspective view of a fracture stabilization orthosis having two stabilizing foam pads or pieces on an inner or interior surface of the fabric or fabric wrap according to an embodiment of the present invention.

FIG. 6a is a side view of a fracture stabilization orthosis having a footplate according to an embodiment of the present invention fitted to a human lower leg. FIG. 6b is an elevated perspective view of the orthosis of FIG. 6a.

### DETAILED DESCRIPTION OF INVENTION

The present invention is directed to a non-invasive fracture stabilization orthosis. In particular embodiments, the fracture stabilization orthosis may help stabilize tibia and fibula bone fractures of the lower leg. The orthosis may also be useful, however, for other limb bone fractures (e.g., of the arm or upper leg).

In this detailed description, references to “one embodiment”, “an embodiment”, or “in embodiments” mean that the feature being referred to is included in at least one embodiment of the invention. Moreover, separate references to “one embodiment”, “an embodiment”, or “embodiments” do not necessarily refer to the same embodiment; however, neither are such embodiments mutually exclusive, unless so stated, and except as will be readily apparent to those skilled in the art. Thus, the invention can include any variety of combinations and/or integrations of the embodiments described herein.

As used herein “substantially”, “generally”, “about”, and other words of degree are relative modifiers intended to indicate permissible variation from the characteristic so modified (e.g., ±0.1%, ±0.5%, ±1.0%, ±2%, ±5%, ±10%, ±20%). It is not intended to be limited to the absolute value or characteristic which it modifies but rather possessing more of the physical or functional characteristic than its opposite, and preferably, approaching or approximating such a physical or functional characteristic.

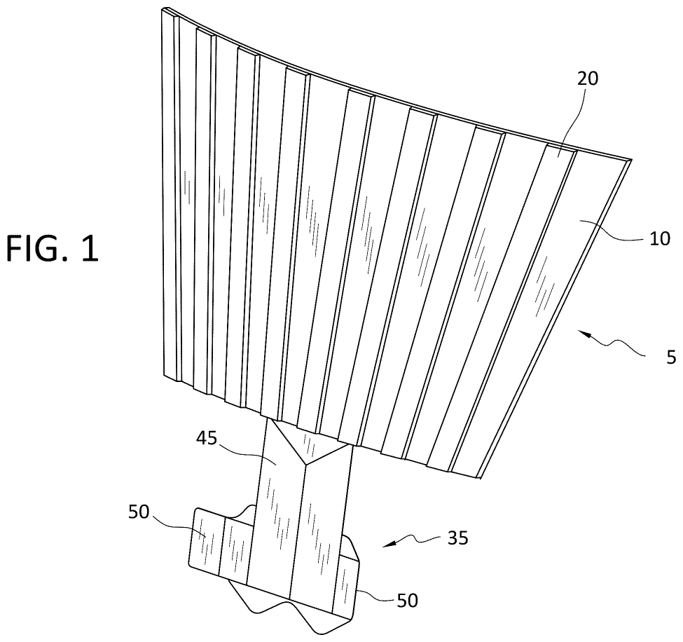

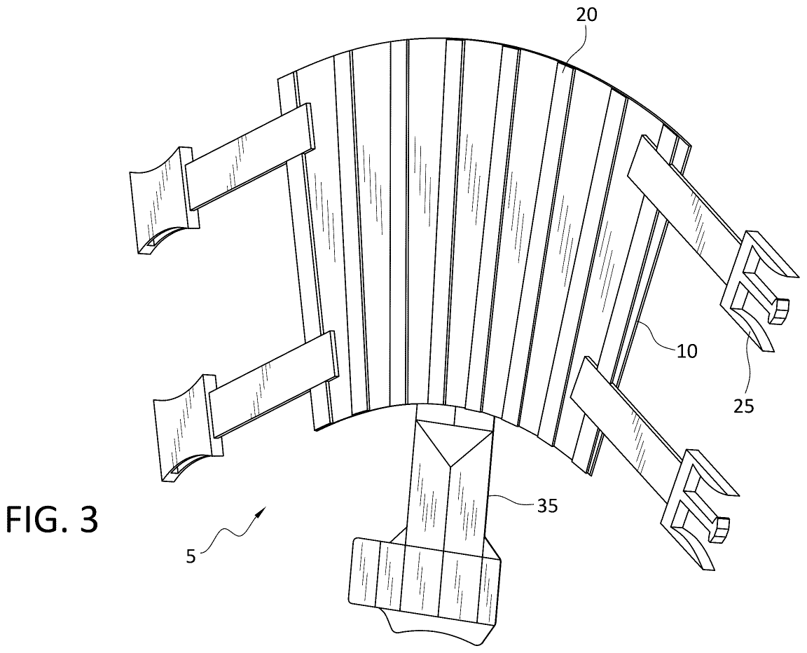

As shown in FIGS. 1-4, the fracture stabilization orthosis 5 of the present invention comprises a fabric or fabric wrap 10 reinforced with a plurality of rigid and/or light weight struts 15 within pockets or sleeves 20. The fabric or fabric wrap 10 may comprise any fabric having a desired elasticity. In embodiments, the fabric may be NEOPRENE®, RIPSTOCK NYLON®, GORE-TEX®, or SPANDEX®. In embodiments, the orthosis 5 may include a fastener 25 for affixing it to a limb, such as a lower leg. The fastener may be one or more of a belt, strap, buckle, zipper, snap, or a hook-and-loop fastener such as VELCRO®.

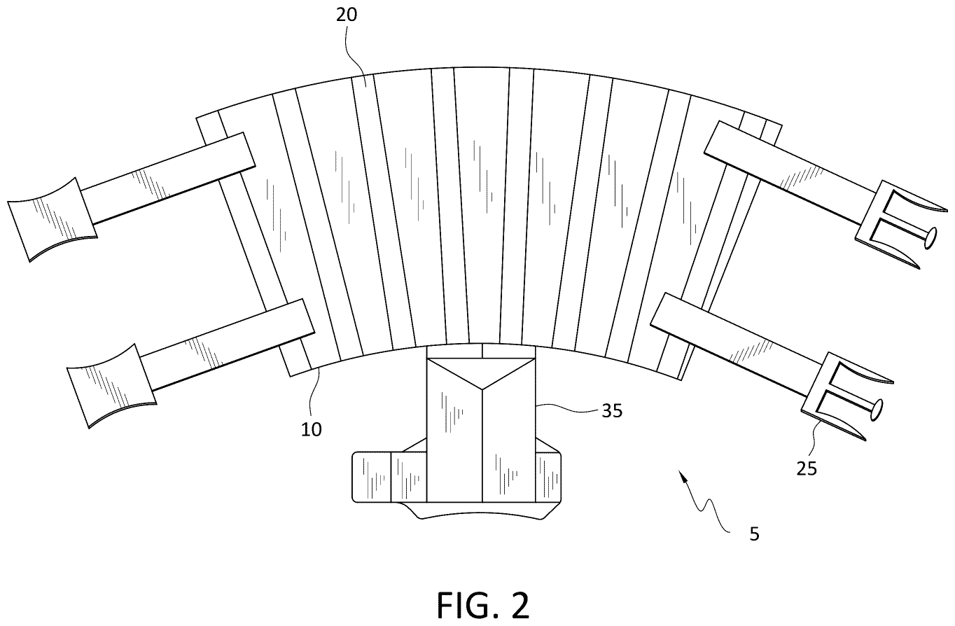

When laid flat as shown in FIG. 2, in embodiments, an upper edge of the fabric or fabric wrap may have a convex shape and a lower edge of the fabric or fabric wrap may also have a convex shape.

The fabric or fabric wrap 10 may be shaped so that, when wrapped in use around a limb, it forms a frustum, for example a cone-shaped frustrum. In embodiments, the frustum may approximate a lower leg anatomy and conform to the lower leg. The orthosis may be scaled to fit anyone. In a particular embodiment, the orthosis may be scaled to a US army soldier population from the fifth percentile female through ninety-fifth percentile male by lower limb size.

In an embodiment, the frustum has a pitch that approximates that of a wearer (e.g., patient, soldier, service provider, defense service member, and the like). In specific embodiments, the pitch of the frustum when in use around the lower leg is about 0.6 to about 0.7, for example, 0.66. Pitch may be defined as (Calf Circumference−Ankle Circumference)/(Shank Length). Shank Length is Calf Height-Lateral Malleoli Height, wherein Calf Height is the distance from a part of the calf with the largest circumference to the ground.

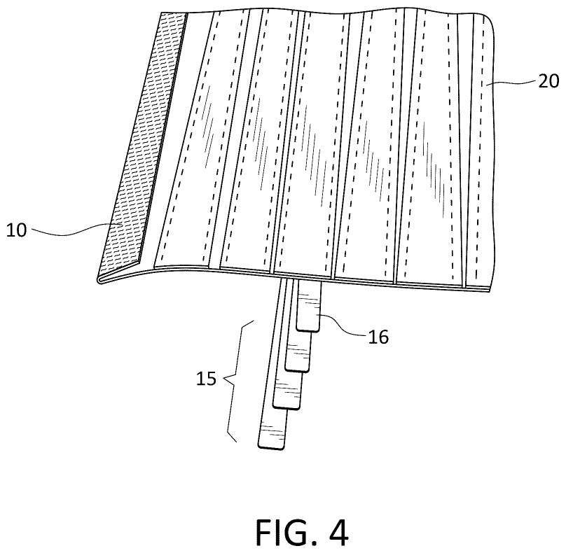

The plurality of struts 15 provide stability to help immobilize a limb with a bone fracture. In embodiments, the plurality of struts 15 may be, for example, about 4 to about 12 struts (e.g., FIG. 4). The struts 15 within pockets or sleeves 20 may be equally spaced along or around the fabric or fabric wrap 10. The struts 15 may be sewn into the fabric or fabric wrap 10 or may be removably insertable into the pockets or sleeves 20 that substantially extend a length of the fabric or fabric wrap (e.g., a length of a lower leg).

In specific embodiments, each strut may have a width of about 0.5 cm to about 2.0 cm. The width of the pocket or sleeves may be about 1.5 cm to about 3.5 cm, for example, about 2.0 cm to about 3.0 cm. Each strut may have a length of about 30 cm to about 50 cm, for example, about 45 cm.

In specific embodiments, as shown in FIG. 4, each strut 15 may comprise at least one, at least two, or three or more, for example four to eight, sheets of material 16 that can conform to the contours of the leg, while providing rigidity in other planes. The sheets 16 may be the same material or different materials and may comprise, but are not limited to, aluminum, titanium, steel, nylon, carbon fiber laminate, fiberglass laminate, KEVLAR® laminate, ultra-high-molecular-weight polyethylene (UHMWPE or DYNEEMA®), carbon fiber (e.g., a unidirectionally woven carbon fiber) or a laminated composite of any combination thereof. Any material with the required degree of low stiffness and high ductility may be used that allows the fracture stabilization orthosis to accommodate the shape of the lower limb (e.g., over the fibular head or non-uniform shapes due to the injury), but then act to make the combination of the lower leg and the orthosis stiffer when the orthosis is tightened, thus stabilizing an injured or fracture site. In embodiments, the struts may be pliable so they are bendable away from a limb, for example, so that the orthosis may be fit to people with short lower legs. In embodiments, the sheets of material 16 may be bound together via an adhesive material. In specific embodiments, a thickness of each sheet may be about 0.25 mm to about 2 mm, for example, 0.4 mm to about 1 mm.

In a specific embodiment, the fracture stabilization orthosis may comprise about two to about twelve struts 15, each strut comprising two sheets 16. In another embodiment, the fracture stabilization orthosis may comprise about six struts, each strut comprising three sheets or four sheets. In embodiments, each strut may comprise at least one sheet of ultra-high-molecular-weight polyethylene (DYNEEMA®), at least one sheet of aluminum, at least one sheet of a unidirectionally woven carbon fiber or fiberglass, or any combination thereof.

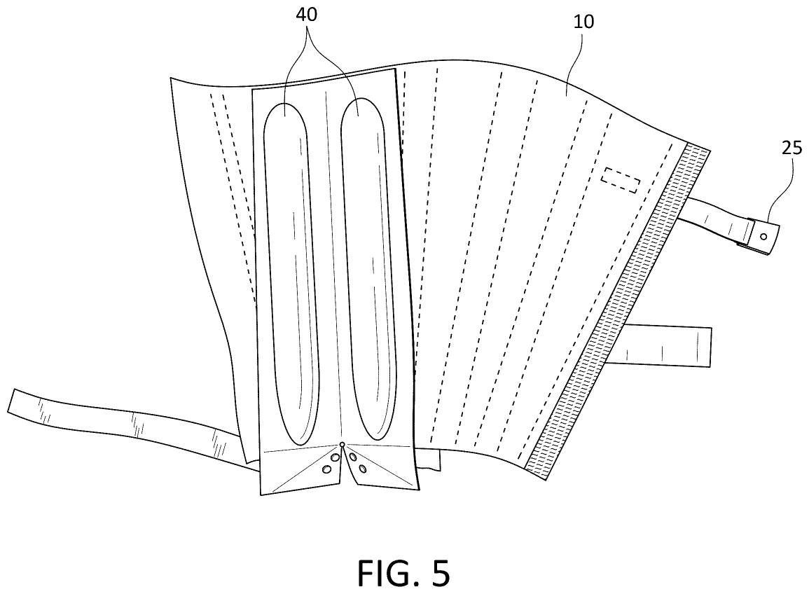

In embodiments, as shown in FIG. 5, the fabric or fabric wrap 10 may include two stabilizing pads or pieces of foam 40 on an inner surface thereof, so that when the orthosis is wrapped around a lower leg, each foam piece rests along an opposing side of the tibia, that is, each piece of foam extends along an opposing side of the tibia crest. The stabilizing pads may be wide enough so as not to put direct pressure on the tibia or fibula bones. In specific embodiments, the pads or pieces of foam may have a length of about 11 inches to 14 inches, a width of about 1 inch to about 2 inches, and a convex cross section having a maximum thickness of about ⅜ inch to about 0.5 inch.

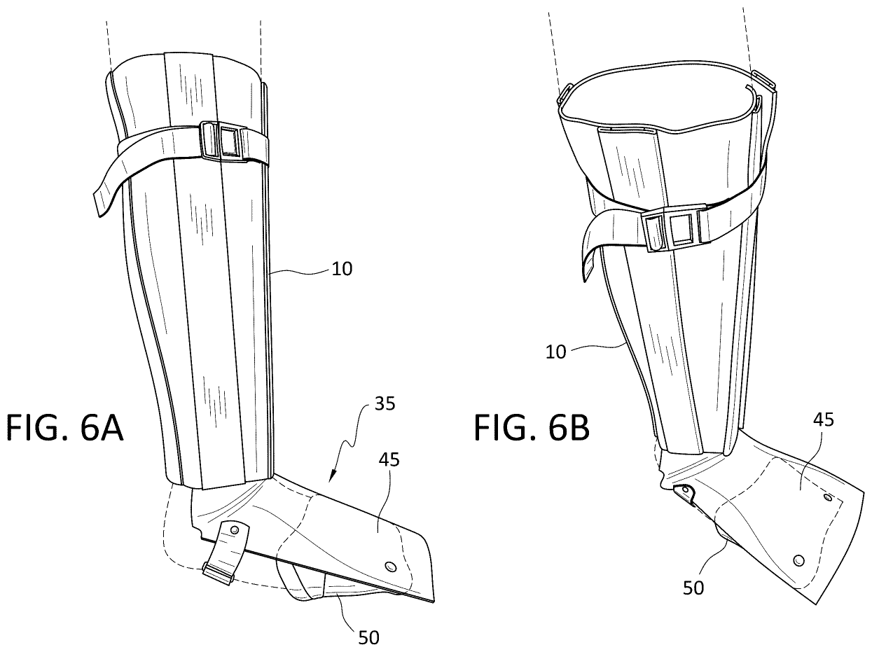

In specific embodiments, and as shown in FIGS. 1-3 and 6a-6b, the orthosis may have a footplate 35 that is permanently connected or removably connectable to the fabric or fabric wrap 10. The footplate 35 may be made of a plastic, such as polyethylene. In a specific embodiment, the footplate 35 may comprise a rectangular-shaped piece 45 that may be foldable so that it has a curvature that protects a top surface of the foot. In embodiments, the footplate 35 may also have one or more sections 50 extending outwardly from the rectangular-shaped piece 45 and which may be wrapped around the foot to provide padding (e.g., as a padded footwrap). In a specific embodiment, the orthosis may also have an ankle section and/or a fastener for securing the footplate 35 around a bottom of the foot and/or ankle.

### INDUSTRIAL APPLICABILITY

The present invention is directed to a fracture stabilization orthosis having a fabric or fabric wrap and a plurality of struts. The fracture stabilization orthosis may be affixed to a limb of a person having a bone fracture to provide increased mobility.

Although the present invention has been described in terms of particular exemplary and alternative embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

What is claimed is:

1. A fracture stabilization orthosis, comprising:
a fabric or fabric wrap; and
a plurality of struts, each strut comprising two or more sheets of a material conformable to contours of a leg and insertable into a sleeve or pocket on the fabric or fabric wrap,
wherein the fabric or fabric wrap is shaped so that when it is wrapped around a limb it forms a frustum.

2. The fracture stabilization orthosis of claim 1, comprising about 4 to about 12 vertical struts substantially extending a length of the fabric or fabric wrap.

3. The fracture stabilization orthosis of claim 1, wherein each strut comprises a plurality of layered sheets.

4. The fracture stabilization orthosis of claim 1, wherein each strut comprises four to eight sheets of material.

5. The fracture stabilization orthosis of claim 1, wherein the fabric or fabric wrap comprises an elastic material.

6. The fracture stabilization orthosis of claim 1, wherein each strut comprises two or more sheets, each sheet comprising aluminum, titanium, steel, nylon, carbon fiber laminate, fiberglass laminate, KEVLAR® laminate, ultra-high-molecular-weight polyethylene, or a laminated composite of any combination thereof.

7. The fracture stabilization orthosis of claim 1, wherein a thickness of each sheet may be about 0.25 mm to about 2 mm.

8. The fracture stabilization orthosis of claim 1, comprising about six struts, each strut comprising about three to about four sheets.

9. The fracture stabilization orthosis of claim 8, wherein each strut comprises at least one sheet of ultra-high-molecular-weight polyethylene, at least one sheet of carbon fiber; or at least one sheet of aluminum.

10. The fracture stabilization orthosis of claim 1, wherein each strut has a length of about 30 cm to about 50 cm and a width of about 0.5 cm to about 2.0 cm.

11. The fracture stabilization orthosis of claim 1, further comprising at least one fastener for affixing the orthosis to a limb, wherein the at least one fastener is one or more of a belt, strap, buckle, zipper, snap, or a hook-and-loop fastener.

12. The fracture stabilization orthosis of claim 1, further comprising two pieces of foam on an inner surface of the fabric or fabric wrap, so that when the orthosis is wrapped around a lower leg, each foam piece rests along an opposing side of the tibia crest.

13. The fracture stabilization orthosis of claim 1, wherein the orthosis is configured to completely enclose a calf of a wearer.

14. The fracture stabilization orthosis of claim 1, wherein the frustum has a pitch of about 0.6 to about 0.7.

15. The fracture stabilization orthosis of claim 1, wherein the frustum has a pitch of 0.66.

16. The fracture stabilization orthosis of claim 1, wherein the orthosis further comprises a footplate connected to a bottom of the fabric or fabric wrap.

17. The fracture stabilization orthosis of claim 16, wherein the footplate is foldable to substantially cover a top surface of a foot.

18. The fracture stabilization orthosis of claim 16, wherein the footplate further comprises one or more sections that provide padding and are wrappable around at least part of the foot.

19. A method of stabilizing a fracture, comprising:
providing the fracture stabilization orthosis of claim 1; and
wrapping the fracture stabilization orthosis around a limb of a person having a bone fracture.

20. A method of stabilizing a fracture of claim 19, wherein the bone fracture is a tibia or fibula bone fracture.
